(19) United States
(12) Patent Application Publication
(10) Pub. No.: US 2006/0148002 A1
(43) Pub. Date: Jul. 6, 2006

(54) Methods and compositions for the treatment and diagnosis of body weight disorders

(76) Inventors: Wei Gu, Brookline, MA (US); Liang-Ping Weng, Newton, MA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/370,754

(22) Filed: Mar. 8, 2006

Related U.S. Application Data
(63) Continuation of application No. 10/186,499, filed on Jul. 1, 2002, now abandoned.
(60) Provisional application No. 60/303,266, filed on Jul. 5, 2001.

(51) Int. Cl. G01N 33/53; A61K 38/17

(52) U.S. Cl. 435/7.1; 514/12

(57) ABSTRACT

The invention relates to methods and compositions for the treatment and diagnosis of body weight disorders, including, but not limited to, obesity, overweight, anorexia, cachexia, insulin resistance, and diabetes. The invention further provides methods for identifying a compound capable of treating a body weight disorder. In addition, the invention provides a method for treating a subject having a body weight disorder characterized by aberrant 58128 polypeptide activity or aberrant 58128 nucleic acid expression. In another aspect, the invention provides methods for modulating 58128 polypeptide activity or 58128 expression in a subject.

### CROSS-REFERENCES TO RELATED APPLICATIONS

This application is a continuation of Ser. No. 10/186,499 filed on Jul. 1, 2002 which claims the benefit of U.S. Provisional Application No. 60/303,266, filed Jul. 5, 2001, the contents of each are herein incorporated by reference.

### BACKGROUND OF THE INVENTION

The G-protein-coupled receptors (GPCR) form an important class of peptide-binding receptors. The various members of the GPCR family mediate a wide variety of intercellular signals.

Members of the GPCR family have seven helical domains which span the cell membrane and are linked by three extracellular loops and three intracellular loops. The receptors also possess an extracellular amino terminal tail and an intracellular carboxy terminal tail. The intracellular loops interact with a G-protein that can switch from a GDP-binding form to a GTP-binding form.

The binding of an appropriate ligand to a GPCR initiates the conversion of the coupled G-protein from its GDP-binding form to its GTP-binding form. This conversion, in turn, initiates a signal transduction cascade that generates a biological response. Depending on the nature of the GPCR, signal transduction activity can be measured by measuring the intracellular Ca2+ source level, phospholipase C activation, the inositol triphosphate (IP3) level, the diacylglycerol level, or the adenosine cyclic 3′,5′-monophosphate (AMP) level.

Lee et al. (2000, Biochim. Biophys. Acta 1490:311-323) describes the cloning and characterization of additional members of the biogenic amine receptor subfamily of GPCRS, including GPR58.

Obesity represents the most prevalent of body weight disorders with estimates ranging from 30% to 50% within the middle-aged population in the western world. Other body weight disorders, such as anorexia nervosa and bulimia nervosa which together affect approximately 0.2% of the female population of the western world, also pose serious health threats. Further, such disorders as anorexia and cachexia (wasting) are also prominent features of other diseases such as cancer, cystic fibrosis, and AIDS.

Obesity, defined as a body mass index (BMI) of 30 kg/m or more, also contributes to other diseases. For example, this disorder is responsible for increased incidences of diseases such as coronary artery disease, hypertension, stroke, diabetes, hyperlipidemia and some cancers. (See, e.g., Nishina, P. M. et al. (1994) Metab. 43:554-558; Grundy, S. M. & Barnett, J. P. (1990), Dis. Mon. 36:641-731). Obesity is a complex multi-factorial chronic disease that develops from an interaction of genotype and the environment. The development of obesity involves social, behavioral, cultural, physiological, metabolic and genetic factors.

Generally, obesity results when energy intake exceeds energy expenditure. Increasing energy expenditure thus is an important strategy for decreasing body weight.

Given the prevalence of obesity and other body weight disorders, there currently exists a great need for methods and compositions which can modulate body weight, body fat, and/or metabolic rate, and which can therefore treat such disorders.

### SUMMARY OF THE INVENTION

The invention provides assays for the identification of compounds useful for the modulation of body weight. Such compounds are useful for the treatment of body weight disorders, including, but not limited to, obesity, overweight, anorexia, cachexia, diabetes, and insulin resistance. The methods of the invention include cell-free and cell-based assays that identify compounds (modulators) which bind to and/or activate or inhibit the activity or expression of 58128, a G protein-coupled receptor, and in vivo assays to measure the effect of the compound on feeding behavior, body weight, body fat, or metabolic rate. The invention also provides compounds which bind to and/or activate or inhibit the activity of 58128 as well as pharmaceutical compositions comprising such compounds.

Accordingly, the invention provides methods for the diagnosis and treatment of disorders or diseases including but not limited to obesity, overweight, anorexia, cachexia, diabetes, and insulin resistance.

In one aspect, the invention provides methods for identifying a compound capable of treating a body weight disorder, e.g., obesity, overweight, anorexia, cachexia, diabetes, or insulin resistance. The method includes assaying the ability of the compound to modulate 58128 nucleic acid expression or 58128 polypeptide activity.

In another aspect, the invention features a method for treating a subject having a body weight disorder characterized by aberrant 58128 polypeptide activity or aberrant 58128 nucleic acid expression, e.g., obesity, overweight, anorexia, cachexia, diabetes, or insulin resistance. The method includes administering to the subject a 58128 modulator, e.g., in a pharmaceutically acceptable formulation or by using a gene therapy vector. In one embodiment, the 58128 modulator may be a small molecule, an anti-58128 antibody, a 58128 polypeptide comprising the amino acid sequence of SEQ ID NO:2 or 5, or a fragment thereof, a 58128 polypeptide comprising an amino acid sequence which is at least 90 percent identical to the amino acid sequence of SEQ ID NO:2 or 5, an isolated naturally occurring allelic variant that encodes a polypeptide consisting of the amino acid sequence of SEQ ID NO:2 or 5, an antisense 58128 nucleic acid molecule, a nucleic acid molecule of SEQ ID NO:1, 3, 4, or 6, or a fragment thereof, or a ribozyme.

The invention also features pharmaceutical compositions comprising a compound identified using the screening methods of the invention as a well as methods for preparing such compositions by combining a such a compound and a pharmaceutically acceptable carrier. Also within the invention are pharmaceutical compositions comprising a compound identified using the screening assays of the invention packaged with instructions for use. For modulators that are antagonists of 58128 activity or expression, the instructions specify use of the pharmaceutical composition for treatment of high body weight (e.g., for reduction of body weight). For modulators that are agonists of 58128 activity or expression, the instructions specify use of the pharmaceutical composition for treatment of low body weight (i.e., for increase of body weight).

In addition, the invention includes nucleic acid molecules comprising a nucleotide sequence encoding all or a portion of 58128, polypeptides comprising all or a portion of 58128, antibodies directed against 58128, mammals harboring a 58128 transgene (e.g., mice expressing 58128 overexpressing the murine orthologue), and mammals in which the expression of a naturally occurring allelic variant of the 58128 gene has been deleted or mutated.

Other features and advantages of the invention will be apparent from the following detailed description and claims.

### DETAILED DESCRIPTION OF THE INVENTION

The invention provides methods and compositions for the diagnosis and treatment of body weight disorders. The invention is based, at least in part, on the discovery that expression of the 58128 gene is limited to the brain, more specifically to regions of the brain associated with regulation of feeding behavior, e.g., the hypothalamus. 58128, also referred to herein as GPR58 (GenBank Accession No. AF112460), is a member of a biogenic amine-like receptor subfamily of GPCRs which includes GPR57, putative neurotransmitter receptor (PNR), and a 5-HT4 pseudogene (Lee, D. K., et al (2000) Biochim. Biophys. Acta 1490:311-323).

The methods of the invention include identifying candidate or test compounds or agents (e.g., peptides, polypeptides, peptidomimetics, and small molecules) which interact with (e.g., bind) 58128, and/or modulate the activity or expression of 58128. Thus, 58128 modulators are useful in the treatment of body weight disorders, e.g., obesity, overweight, cachexia, and anorexia. Moreover, modulators of 58128 can also be effective in the treatment of diabetes caused by insulin resistance.

As used herein, a “body weight disorder” includes a disease, disorder, or condition which is associated with abnormal or aberrant body weight or percentage of body fat. Body weight disorders can be characterized by a misregulation (e.g., downregulation or upregulation) of 58128 activity. Examples of body weight disorders include disorders such as obesity, overweight, anorexia, and cachexia. Obesity is defined as a body mass index (BMI) of 30 kg/²m or more (National Institute of Health, Clinical Guidelines on the Identification, Evaluation, and Treatment of Overweight and Obesity in Adults (1998)). However, the invention is also intended to include a disease, disorder, or condition that is characterized by a body mass index (BMI) of 25 kg/²m or more, 26 kg/²m or more, 27 kg/²m or more, 28 kg/²m or more, 29 kg/2m or more, 29.5 kg/2m or more, or 29.9 kg/²m or more, all of which are typically referred to as overweight (National Institute of Health, Clinical Guidelines on the Identification, Evaluation, and Treatment of Overweight and Obesity in Adults (1998)). Body weight disorders also include conditions or disorders which are secondary to disorders such as obesity or overweight, i.e., are influenced or caused by a disorder such as obesity or overweight. For example, insulin resistance, diabetes, hypertension, and atherosclerosis can all be influenced or caused by obesity or overweight. Accordingly, such secondary conditions or disorders are additional examples of body weight disorders as defined herein.

As used interchangeably herein, “58128 activity,” “biological activity of 58128” or “functional activity of 58128,” includes an activity exerted by a 58128 protein, polypeptide, or nucleic acid molecule on a 58128 responsive cell or tissue or on a 58128 substrate or ligand, e.g., a protein substrate or ligand, as determined in vivo or in vitro, according to standard techniques. 58128 activity can be a direct activity, such as an association with a 58128 target molecule. As used herein, a “target molecule” or “binding partner” is a molecule with which a 58128 protein binds or interacts in nature, such that a 58128-mediated function, is achieved. In an exemplary embodiment, the target molecule is a 58128 ligand. A 58128 target molecule can be a non-58128 molecule (e.g., NAD⁺, NADP⁺, or other cofactor) or a 58128 protein or polypeptide. Examples of such target molecules include proteins in the same signal transduction pathway as the 58128 protein, e.g., proteins which may function upstream (including both stimulators and inhibitors of activity) or downstream of the 58128 protein in a 58128-mediated signal transduction pathway. Alternatively, a 58128 activity is an indirect activity, e.g., a cellular signaling activity mediated by interaction of the 58128 protein with a 58128 target molecule. The biological activities of 58128 are described herein. For example, the 58128 proteins can have one or more of the following activities: 1) the ability to modulate metabolism or catabolism of biochemical molecules (e.g., molecules involved in modulating a nerve cell activity); 2) the ability to sense and mediate cellular response to environmental stimuli, e.g., small molecules or protein ligands; 3) the ability to sense and mediate cellular response to biological messengers, e.g., secreted hormones; or 4) the ability to signal to G proteins.

Additionally, the 58128 molecules of the invention can have one or more of the following activities: 1) the ability to regulate, sense and/or transmit an extracellular signal into a cell, for example, a nerve cell; 2) the ability to interact with (e.g., bind to) an extracellular signal or a cell surface receptor; 3) the ability to mobilize an intracellular molecule that participates in a signal transduction pathway (e.g., adenylate cyclase or phosphatidylinositol 4,5-bisphosphate (PIP₂), inositol 1,4,5-triphosphate (IP₃)); 5) the ability to control production or secretion of molecules; 6) the ability to alter the structure of a cellular component; 7) the ability to modulate cell proliferation, e.g., synthesis of DNA; and 8) the ability to modulate cell migration, cell differentiation; and cell survival e.g., of the cells or tissue in which they are expressed (e.g., hypothalarnus, in particular, the ventral/medial hypothalamus, as described in the exemplification). Thus, the 58128 molecules can act as novel diagnostic targets and therapeutic agents in the treatment of GPCR associated disorders.

As used herein, a “signaling transduction pathway” refers to the modulation (e.g., stimulation or inhibition) of a cellular function/activity upon the binding of a ligand to its receptor, e.g., a GPCR. Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase. 58128 is a GPCR and, thus, interacts with G proteins to produce one or more secondary signals, in a variety of intracellular signal transduction pathways, e.g., through phosphatidylinositol or cAMP metabolism and turnover, in a cell.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of ligand to the receptor activates, in some cells, the plasma membrane enzyme phospholipase C that, in turn, can hydrolyze PIP₂ to produce 1,2-diacylglycerol (DAG) and IP₃. Once formed, IP₃ can diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which stimulates calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell and used to synthesize PIP₂. The other second messenger produced by the hydrolysis of PIP₂, namely DAG, remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it may be activated by DAG. The activation of protein kinase C in different cells results in various cellular responses such as the phosphorylation of glycogen synthase, or the phosphorylation of various transcription factors, e.g., NF-κB. The language “phosphatidylinositol activity”, as used herein, refers to an activity of PIP₂ or one of its metabolites.

Another signaling pathway in which the receptor can participate is the cAMP turnover pathway. As used herein, “cyclic AMP turnover and metabolism” refers to the molecules involved in the turnover and metabolism of cAMP, as well as to the activities of these molecules. Cyclic AMP is a second messenger produced in response to ligand-induced stimulation of certain GPCRs. In the cAMP signaling pathway, binding of a ligand to a GPCR can lead to the activation of the adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

The 58128 molecules, and modulators thereof, can act as novel therapeutic agents for treating one or more of GPCR associated disorders, e.g., disorders encompassing a central nervous system (CNS) function involved in the regulation of body weight or body fat metabolism as described herein.

Various aspects of the invention are described in further detail in the subsections below.

Screening Assays

The invention provides methods (also referred to herein as “screening assays”) for identifying modulators, i.e., candidate or test compounds or agents (e.g., including peptides, proteins and antibodies, and fragments thereof, peptidomimetics, small molecules, ribozymes, and 58128 antisense molecules) which bind to 58128 proteins, have a stimulatory or inhibitory effect on 58128 expression or 58128 activity, or have a stimulatory or inhibitory effect on the expression or activity of a 58128 target molecule. Compounds identified using the assays described herein are useful for treating body weight disorders.

Candidate or test compounds or agents which interact with (e.g., bind) 58128 and/or have a stimulatory or inhibitory effect on the activity or the expression of 58128 are identified in assays that employ either cell-based assays using cells which express a form of 58128 or cell-free assays using a form of isolated 58128. The various assays can employ any of a variety of forms of 58128 (e.g., full-length 58128, a biologically active fragment of 58128, or a fusion protein which includes all or a portion of 58128). Moreover, the 58128 can be derived from any suitable mammalian species (e.g., human, rat, mouse, monkey), e.g., including, but not limited to, human 58128, rat 58128, and murine 58128. The assay can be a binding assay using direct or indirect measurement of the binding of a test compound or a 58128 ligand to 58128 itself. Alternatively, the assay can be an assay using direct or indirect measurement of a biological activity of 58128. The assay can also be an expression assay using direct or indirect measurement of the expression of 58128 (e.g., mRNA encoding a 58128 protein, or fragment thereof). Additionally, the various screening assays can be combined with an in vivo assay of the effect of the test compound on the feeding behavior, body weight, body fat, or metabolic rate of a suitable mammal (e.g., including, but not limited to, a mouse or a rat).

In one aspect, the assay is a cell-based assay in which a cell expressing a membrane-bound form of a 58128 protein, e.g., a full length 58128, a biologically active fragment of 58128, or a fusion protein which includes all or a fragment of 58128, (e.g., a brain cell or a cell transfected with a nucleic acid molecule encoding a 58128 protein, e.g., SEQ ID NO:2 or 5, or fragment thereof) is contacted with a test compound, and the ability of the test compound to modulate 58128 activity is determined. In a preferred embodiment, the biologically active fragment of the 58128 protein includes a domain or motif which can modulate a GPCR activity, e.g., alter intracellular Ca²⁺ concentration, activate phospholipase C, alter intracellular IP₃ concentration, alter intracellular DAG concentration, and alter intracellular cAMP concentration. Alternatively, determining the ability of the test compound to modulate 58128 activity can be accomplished by monitoring, for example, the production of one or more specific metabolites (e.g., ¹⁴C-glucose) or replacement of nutrients in a cell which expresses a 58128 protein (see, e.g., Saada et al. (2000) Biochem. Biophys. Res. Commun. 269:382-386).

The ability of a test compound to modulate insulin sensitivity of a cell can be determined by performing an assay in which cells that express 58128, e.g., brain cells, are contacted with the test compound, e.g., transformed to express the test compound; incubated with radioactively labeled glucose (¹⁴C-glucose); and treated with insulin. An increase or decrease in ¹⁴C-glucose in the cells containing the test compound as compared to control cells indicates that the test compound can modulate insulin sensitivity of the cells. Alternatively, the cells containing the test compound can be incubated with a radioactively labeled phosphate source (e.g., [³²P]ATP) and treated with insulin. Phosphorylation of proteins in the insulin pathway, e.g., the insulin receptor, can then be measured. An increase or decrease in phosphorylation of a protein in the insulin pathway in cells containing the test compound as compared to the control cells indicates that the test compound can modulate insulin sensitivity of the cells.

In another aspect, determining the ability of the test compound to modulate the activity of 58128 can be achieved, for example, by determining the ability of 58128 to bind to or interact with a target molecule. The target molecule can be a molecule with which 58128 binds or interacts with in nature, for example, a molecule on the surface of a cell which co-expresses 58128, a molecule on the surface of a second cell, a molecule in the extracellular milieu, a molecule associated with the internal surface of a cell membrane, or a cytoplasmic molecule. The target molecule can be a component of a signal transduction pathway which facilitates transduction of an extracellular signal (e.g., a signal generated by binding of a 58128 ligand to 58128) through the cell membrane and into the cell. The target molecule can be, for example, a second intracellular protein which has catalytic activity or a protein which facilitates the association of downstream signaling molecules with 58128.

Determining the ability of a 58128 polypeptide to bind to or interact with a target molecule can be accomplished by any of the methods described herein for determining direct binding. In one embodiment, determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca²⁺, DAG, IP₃, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response.

In a competitive binding format, the assay includes contacting a 58128-expressing cell (e.g., a brain cell or a cell transfected with a nucleic acid molecule encoding a 58128 protein, e.g., SEQ ID NO:2 or 5, or a fragment thereof) with a compound known to bind 58128 to form an assay mixture, contacting the assay mixture with a test compound, and determining the ability of the test compound to compete with the known compound to interact with or bind the 58128-expressing cell. Thus, the ability of the test compound to interact with the 58128-expressing cell is measured by determining the ability of the 58128-expressing cell to preferentially bind the test compound in the presence of the known compound.

To determine whether a test compound modulates 58128 expression, a cell which expresses 58128 (e.g., a brain cell or a cell transfected with a nucleic acid molecule encoding a 58128 protein, e.g., SEQ ID NO:2 or 5, or a fragment thereof) is contacted with a test compound, and the ability of the test compound to modulate 58128 expression is determined by measuring 58128 mRNA by, e.g., Northern Blotting, quantitative PCR (e.g., TaqMan), or in vitro transcriptional assays. To perform an in vitro transcriptional assay, the full length promoter and enhancer of 58128 can be linked to a reporter gene, such as chloramphenicol acetyltransferase (CAT) or luciferase, and introduced into host cells. The same host cells are then transfected with or contacted with the test compound. The effect of the test compound can be measured by reporter gene activity and then compared to reporter gene activity in cells which do not contain the test compound. A difference, e.g., an increase or decrease, in reporter gene activity relative to activity in cells which do not contain the test compound therefore indicates a modulation of 58128 expression by the test compound.

Alternatively, modulators of 58128 expression can be identified using a method in which a cell is contacted with a candidate compound and the expression of 58128 protein or 58128 mRNA in the cell is determined. The level of expression of 58128 protein or mRNA in the presence of the candidate compound is compared to the level of expression of 58128 protein or 58128 mRNA in the absence of the candidate compound. The candidate compound is then identified as a modulator of expression of 58128 based on this comparison. For example, when expression of 58128 protein or mRNA protein is greater (i.e., statistically significantly greater) in the presence of the candidate compound than in its absence, the candidate compound is identified as a stimulator or agonist of 58128 protein synthesis or mRNA expression. Alternatively, when expression of 58128 protein or mRNA is less (i.e., statistically significantly less) in the presence of the candidate compound than in its absence, the candidate compound is identified as an inhibitor or antagonist of 58128 protein synthesis or mRNA expression. The level of 58128 protein or mRNA in the cells can be determined by any of the methods described herein.

In another embodiment, an assay of the invention is a cell-free assay in which a 58128 protein, or biologically active portion thereof, is contacted with a test compound, and the ability of the test compound to bind or modulate (e.g., stimulate or inhibit) the activity of the 58128 protein, or biologically active portion thereof, is determined. Preferred biologically active portions of the 58128 proteins to be used in assays of the invention include fragments which participate in interactions with non-58128 molecules, e.g., fragments with high surface probability scores.

Binding of the test compound to the 58128 protein can be determined either directly or indirectly as described herein. Determining the ability of the 58128 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another aspect, the assay is a cell-free assay in which the ability of a test compound to modulate 58128 interaction (e.g., binding) with a 58128 target molecule (e.g., a 58128 substrate or ligand) is determined. Determining the ability of a test compound to modulate 58128 binding to a substrate can be accomplished, for example, by coupling the 58128 substrate with a radioisotope or fluorescent or enzymatic label such that binding of the 58128 substrate to 58128 can be determined by detecting the presence of the labeled 58128 substrate in a complex. Alternatively, 58128 can be coupled with a radioisotope or enzymatic label to monitor the ability of a test compound to modulate 58128 binding to a 58128 substrate in a complex. Determining the ability of the test compound to bind 58128 can be accomplished, for example, by coupling the compound with a radioisotope or enzymatic label such that binding of the compound to 58128 can be determined by detecting the labeled 58128 compound in a complex. For example, 58128 substrates can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with (e.g., bind) 58128 without the labeling of any of the interactants. For example, a microphysiometer can be used to detect the interaction of a compound with 58128 without the labeling of either the compound or the 58128 (McConnell, H. M. et al. (1992) Science 257:1906-1912). As used herein, a “microphysiometer” (e.g., Cytosensor®; Molecular Devices Corp., Sunnyvale Calif.) is an analytical instrument that measures the rate at which a cell acidifies its environment using a light-addressable potentiometric sensor (LAPS). Changes in this acidification rate can be used as an indicator of the interaction between acompound and 58128.

In yet another embodiment, the cell-free assay involves contacting a 58128 protein, or biologically active portion thereof, with a known compound which binds the 58128 protein to form an assay mixture, contacting the assay mixture with a test compound, and determining the ability of the test compound to interact with the 58128 protein, wherein determining the ability of the test compound to interact with the 58128 protein comprises determining the ability of the 58128 protein to preferentially bind to or modulate the activity of a 58128 target molecule (e.g., a 58128 substrate or ligand).

The cell-free assays of the invention are amenable to use of both soluble and/or membrane-bound forms of isolated proteins (e.g., 58128 proteins or biologically active portions thereof ). In the case of cell-free assays in which a membrane-bound form of an isolated protein is used, it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholam idopropyl)dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-1-propane sulfonate.

In more than one embodiment of the above assay methods of the invention, it may be desirable to immobilize either 58128 or a 58128 target molecule to facilitate separation of complexed from uncomplexed forms of one or both of the proteins, as well as to accommodate automation of the assay. Binding of a test compound to a 58128 protein, or interaction of a 58128 protein with a 58128 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST)/58128 fusion proteins or GST/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 58128 protein, and the mixture incubated under conditions conducive to complex formation (e.g., at physiological conditions for salt and pH). Following incubation, the beads or microtitre plates are washed to remove any unbound components, the matrix is immobilized in the case of beads, and complex formation is determined either directly or indirectly, for example, as described above. Alternatively, the complexes can be dissociated from the matrix under appropriate conditions to permit measurement of 58128 binding or activity using standard techniques.

Other techniques for immobilizing proteins or cell membrane preparations on matrices can also be used in the screening assays of the invention. For example, either a 58128 protein or a 58128 target molecule can be immobilized utilizing conjugation of biotin and streptavidin. Biotinylated 58128 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemicals). Alternatively, antibodies which are reactive with 58128 protein or target molecules but which do not interfere with binding of the 58128 protein to its target molecule can be derivatized to the wells of the plate, and unbound target or 58128 protein will be trapped in the wells by antibody conjugation. Methods for detecting such complexes, in addition to those described above for the GST-immobilized complexes, include immunodetection of complexes using antibodies reactive with the 58128 protein or target molecule, as well as enzyme-linked assays which rely on detecting an enzymatic activity associated with the 58128 protein or target molecule.

In another embodiment, the 58128 protein, or fragments thereof, can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and PCT Publication No. WO 94/10300) to identify other proteins which bind to or interact with 58128 (“58128 binding proteins” or “58128 bp”) and are involved in 58128 activity. Such 58128 binding proteins are also likely to be involved in the propagation of signals by the 58128 proteins or 58128 target molecules as, for example, downstream elements of a 58128 mediated signaling transduction pathway. Alternatively, such 58128 binding proteins are inhibitors or antagonists of 58128 activity.

The two-hybrid system is based on the modular nature of most transcription factors, which consist of separable DNA binding and activation domains. Briefly, the assay utilizes two different DNA constructs. In one construct, the gene that codes for a 58128 protein is fused to a gene that encodes the DNA binding domain of a known transcription factor (e.g., GAL-4). In an alternative construct, a DNA sequence, from a library of DNA sequences, that encodes an unidentified protein (“prey” or “sample”) is fused to a gene that encodes the activation domain of the known transcription factor. If the “bait” and the “prey” proteins are able to interact, in vivo, and form a 58128-dependent complex, the DNA binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 58128 protein.

In another aspect, the invention is a combination of two or more of the assays described herein. For example, a modulating agent can be identified using a cell-based or a cell-free assay, and the ability of the agent to modulate the activity of a 58128 protein can be confirmed in vivo, e.g., in an animal such as an animal model for obesity, diabetes, anorexia, or cachexia. Examples of animals that can be used include the transgenic mouse described in U.S. Pat. No. 5,932,779 that contains a mutation in an endogenous melanocortin-4-receptor (MC4-R) gene; animals having mutations which lead to syndromes that include obesity symptoms (described in, for example, Friedman, J. M. et al. (1991) Mamm. Genome 1:130-144; Friedman, J. M. and Liebel, R. L. (1992) Cell 69:217-220; Bray, G. A. (1992) Prog. Brain Res. 93:333-341; and Bray, G. A. (1989) Amer. J. Clin. Nutr. 5:891-902); the animals described in Stubdal H. et al. (2000) Mol. Cell Biol. 20(3):878-82 (the mouse tubby phenotype characterized by maturity-onset obesity); the animals described in Abadie J. M. et al. (2000) Lipids 35:613-20 (the obese Zucker rat (ZR), a genetic model of human youth-onset obesity and type II diabetes mellitus); the animals described in Shaughnessy S. et al. (2000) Diabetes 49:904-11 (mice null for the adipocyte fatty acid binding protein); and the animals described in Loskutoff D. J. et al. (2000) Ann. N.Y. Acad Sci. 902:272-81 (the fat mouse). Other examples of animals that are useful include non-recombinant, non-genetic animal models of obesity such as, for example, rabbit, mouse, or rat models in which the animal has been exposed to long-term over-eating or a high fat diet.

In addition to animal models for obesity, diabetes, cachexia or anorexia, transgenic animals that express a human 58128 can be used to confirm the in vivo effects of a modulator of 58128 identified by a cell-based or cell-free screening assay described herein. Animals of any non-human species, including, but not limited to, mice, rats, rabbits, guinea pigs, pigs, micro-pigs, goats, and non-human primates, e.g., baboons, monkeys, and chimpanzees, may be used to generate 58128 transgenic animals. Alternatively, the transgenic animal comprises a cell, or cells, that includes a gene which misexpresses an endogenous 58128 orthologue such that expression is disrupted, e.g., a knockout animal. Such animals are also useful as a model for studying the disorders which are related to mutated or misexpressed 58128 alleles.

Any technique known in the art may be used to introduce the human 58128 transgene into non-human animals to produce the founder lines of transgenic animals. Such techniques include, but are not limited to, pronuclear microinjection (Hoppe, P. C. and Wagner, 1989, U.S. Pat. No. 4,873,191); retrovirus mediated gene transfer into germ lines (Van der Putten et al. (1985) Proc. Natl. Acad Sci. USA 82:6148-6152); gene targeting in embryonic stem cells (Thompson et al. (1989) Cell 56:313-321); electroporation of embryos (Lo (1983) Mol Cell. Biol. 3:1803-1814); and sperm-mediated gene transfer (Lavitrano et al. (1989) Cell 57:717-723). For a review of such techniques, see Gordon (1989) Transgenic Animals, Intl. Rev. Cytol. 115:171-229, which is incorporated by reference herein in its entirety.

The invention provides for transgenic animals that carry the 58128 transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. ((1992) Proc. Natl. Acad. Sci. USA 89: 6232-6236). The regulatory sequences required for such a cell-type specific activation will depend upon the particular cell type of interest and will be apparent to those of skill in the art. When it is desired that the 58128 transgene be integrated into the chromosomal site of the endogenous 58128 gene, gene targeting is preferred. Briefly, when such a technique is to be utilized, vectors containing nucleotide sequences homologous to the endogenous 58128 gene and/or sequences flanking the gene are designed for the purpose of integrating into, via homologous recombination with chromosomal sequences, and disrupting the function of the endogenous 58128 gene. The transgene may also be selectively expressed in a particular cell type with concomitant inactivation of the endogenous 58128 gene in only that cell type, by following, for example, the teaching of Gu et al. ((1994) Science 265:103-106). The regulatory sequences required for such a cell-type specific recombination will depend upon the particular cell type of interest and will be apparent to those of skill in the art.

Once founder animals have been generated, standard analytical techniques such as Southern blot analysis or PCR techniques are used to analyze animal tissues to determine whether integration of the transgene has taken place. The level of mRNA expression of the transgene in the tissues of the founder animals may also be assessed using techniques which include but are not limited to Northern blot analysis of tissue samples obtained from the animal, in situ hybridization analysis, and RT-PCR. Samples of 58128 gene-expressing tissue, may also be evaluated immunocytochemically using antibodies specific for the 58128 transgene product.

Moreover, a 58128 modulator identified as described herein (e.g., an antisense 58128 nucleic acid molecule, a 58128-specific antibody, or a small molecule) can be used in an animal model to determine the efficacy, toxicity, or side effects of treatment with such a modulator. Alternatively, a 58128 modulator identified as described herein can be used in an animal model to determine the mechanism of action of such a modulator.

Test Compounds

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No.5,223,409), spores (Ladner, supra), plasmids (Cull et al. (1992) Proc. Natl. Acad Sci. USA 89:1865-1869) or phage (Scott and Smith (1990) Science 249:386-390; Devlin (1990) Science 249:404-406; Cwirla et al. (1990) Proc. Natl. Acad Sci. USA 87:6378-6382; Felici (1991) J. Mol. Biol. 222:301 -310; Ladner supra.).

Modeling of Modulators

Computer modeling and searching technologies permit identification of compounds, or an improvement of already identified compounds, that can modulate 58128 expression or activity. Having identified such a compound or composition enables identification of active sites or regions. Such active sites are often ligand binding sites. The active site can be identified using methods known in the art including, for example, from the amino acid sequences of peptides, from the nucleotide sequences of nucleic acids, or from studies of complexes of the relevant compound or composition with its natural ligand. In the latter case, chemical or X-ray crystallographic methods are useful in identifying residues in the active site by locating the position of the complexed ligand.

The three dimensional geometric structure of the active site can be determined using known methods, including X-ray crystallography, from which spatial details of the molecular structure can be obtained. Additionally, solid or liquid phase NMR can be used to determine certain intramolecular distances. Any other experimental method of structure determination known in the art can be used to obtain partial or complete geometric structures. The geometric structures measured with a complexed ligand, natural or artificial, can increase the accuracy of the active site structure determined.

If only an incomplete or insufficiently accurate structure is determined, methods of computer based numerical modeling can be used to complete or improve the accuracy of the structure. Any recognized modeling method may be used, including parameterized models specific to particular biopolymers, such as proteins or nucleic acids, molecular dynamics models based on computing molecular motions, statistical mechanics models based on thermal ensembles, or combined models. For most types of models, standard molecular force fields, which include the forces between constituent atoms and groups, are necessary, and can be selected from force fields known in physical chemistry. The incomplete or less accurate experimental structures can serve as constraints on the complete and more accurate structures computed by these modeling methods.

Having determined the structure of the active site, either experimentally, by modeling, or by a combination of approaches, candidate modulating compounds can be identified by searching databases containing compounds along with information on their molecular structure. Such searches seek compounds having structures that match the determined active site structure and that interact with the groups defining the active site. Such a search can be manual, but is preferably computer assisted. Compounds identified using these search methods can be tested in any of the screening assays described herein to verify their ability to modulate 58128 activity.

Alternatively, these methods can be used to identify improved modulating compounds from an already known modulating compound or ligand. The composition of the known compound can be modified and the structural effects of the modification can be determined by applying the experimental and computer modeling methods described above to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Kaul (1998) Prog. Drug Res. 50:9-105 provides a review of modeling techniques for the design of receptor ligands and drugs. Computer programs that screen and graphically depict chemicals are available from companies such as BioDesign, Inc. (Pasadena, Calif.), Oxford Molecular Design (Oxford, UK), and Hypercube, Inc. (Cambridge, Ontario).

Although described above with reference to design and generation of compounds which can alter binding, one can also screen libraries of known compounds, including natural products or synthetic chemicals, and biologically active materials, including proteins, for compounds which are inhibitors (e.g., antagonists) or activators (e.g., agonists).

Predictive Medicine

The invention also pertains to the field of predictive medicine in which diagnostic assays, prognostic assays, and the monitoring of clinical trials are used for prognostic (or predictive) purposes to thereby treat an individual prophylactically. Accordingly, one aspect of the invention relates to diagnostic assays for determining 58128 protein and/or nucleic acid expression as well as 58128 activity, in the context of a biological sample (e.g., blood, serum, cells, or tissue, e.g., brain tissue) to thereby determine whether an individual is afflicted with a body weight disorder. The invention also provides for prognostic (or predictive) assays for determining whether an individual is at risk of developing a body weight disorder. For example, mutations in a 58128 gene can be assayed in a biological sample. Such assays can be used for prognostic or predictive purpose to thereby prophylactically treat an individual prior to the onset of a body weight disorder.

Another aspect of the invention pertains to monitoring the influence of 58128 modulators (e.g., anti-58128 antibodies or 58128 ribozymes) on the expression or activity of 58128 in clinical trials.

These and other agents are described in further detail in the following sections.

A. Diagnostic Assays For Body Weight Disorders

To determine whether a subject is afflicted with a body weight disorder, a biological sample can be obtained from a subject and the biological sample contacted with a compound or an agent capable of detecting a 58128 protein or nucleic acid (e.g., mRNA or genomic DNA) that encodes a 58128 protein, in the biological sarnple. A preferred agent for detecting 58128 mRNA or genomic DNA is a labeled nucleic acid probe capable of hybridizing to 58128 mRNA or genomic DNA. The nucleic acid probe can be, for example, the 58128 nucleic acid set forth in SEQ ID NO:1, 3, or 4, or a portion thereof, such as an oligonucleotide of at least 15, 20, 25, 30, 25, 40, 45, 50, 100, 250 or 500 nucleotides in length and sufficient to specifically hybridize under stringent conditions to 58128 mRNA or genomic DNA. Other suitable probes for use in the diagnostic assays of the invention are described herein.

A preferred agent for detecting 58128 protein in a sample is an antibody capable of binding to 58128 protein, preferably an antibody with a detectable label. Antibodies can be polyclonal, or more preferably, monoclonal. An intact antibody, or a fragment thereof (e.g., Fab or F(ab′)₂), can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of substances that can be directly coupled to an antibody or a nucleic acid probe include various enzymes, prosthetic groups, fluorescent materials, luminescent materials, bioluminescent materials, and radioactive materials. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 58128 mRNA, protein, or genomic DNA in a biological sample in vitro as well as in vivo. For example, in vitro techniques for detection of 58128 mRNA include Northern hybridizations and in situ hybridizations. In vitro techniques for detection of 58128 protein include enzyme linked immunosorbent assays (ELISAs), Western blots, immunoprecipitations and immunofluorescence. In vitro techniques for detection of 58128 genomic DNA include Southern hybridizations. Furthermore, in vivo techniques for detection of 58128 protein include introducing into a subject a labeled anti-58128 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 58128 protein, mRNA, or genomic DNA, such that the presence of 58128 protein, mRNA or genomic DNA is detected in the biological sample, and comparing the presence of 58128 protein, mRNA or genomic DNA in the control sample with the presence of 58128 protein, mRNA or genomic DNA in the test sample.

B. Prognostic Assays For Body Weight Disorder

The invention further pertains to methods for identifying subjects having or at risk of developing a body weight disorder with aberrant 58128 expression or activity.

As used herein, the term “aberrant” includes a 58128 expression or activity which deviates from the wild type 58128 expression or activity. Aberrant expression or activity includes increased or decreased expression or activity, as well as expression or activity which does not follow the wild type developmental pattern of expression or the subcellular pattern of expression. For example, aberrant 58128 expression or activity is intended to include the cases in which a mutation in the 58128 gene causes the 58128 gene to be under-expressed or over-expressed and situations in which such mutations result in a non-functional 58128 protein or a protein which does not function in a wild type fashion, e.g., a protein which does not interact with a wild type 58128 substrate or ligand, or one which interacts with a non-wild type 58128 substrate or ligand.

The assays described herein, such as the preceding diagnostic assays or the following assays, can be used to identify a subject having or at risk of developing a body weight disorder, e.g., obesity, overweight, anorexia, cachexia, insulin resistance, or diabetes. A biological sample can be obtained from a subject and tested for the presence or absence of a genetic alteration. For example, such genetic alterations can be detected by ascertaining the existence of at least one of: 1) a deletion of one or more nucleotides from a 58128 gene, 2) an addition of one or more nucleotides to a 58128 gene, 3) a substitution of one or more nucleotides of a 58128 gene, 4) a chromosomal rearrangement of a 58128 gene, 5) an alteration in the level of a messenger RNA transcript of a 58128 gene, 6) aberrant modification of a 58128 gene, such as of the methylation pattern of the genomic DNA, 7) the presence of a non-wild type splicing pattern of a messenger RNA transcript of a 58128 gene, 8) a non-wild type level of a 58128-protein, 9) allelic loss of a 58128 gene, and 10) inappropriate post-translational modification of a 58128-protein.

As described herein, there are a large number of assays known in the art which are useful for detecting genetic alterations in a 58128 gene. For example, a genetic alteration in a 58128 gene can be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. NatL Acad. Sci. USA 91:360-364), the latter of which is particularly useful for detecting point mutations in a 58128 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 58128 gene under conditions such that hybridization and amplification of the 58128 gene (if present) occurs, and detecting the presence or absence of an amplification product, or detecting the size of the amplification product and comparing its length to a control sample. In certain situations, PCR and/or LCR are useful as a preliminary amplification step in conjunction with any of the techniques used for detecting mutations described herein.

Alternative amplification methods include, but are not limited to, self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta replicase (Lizardi, P. M. et al. (1988) Bio-Technology 6:1197), as well as any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 58128 gene from a biological sample can be identified by alterations in restriction enzyme cleavage patterns. For example, sample and control DNA are isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 58128 can be identified by hybridizing biological sample derived and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in 58128 can be identified in two dimensional arrays containing light-generated DNA probes as described in Cronin, M. T. et al. (1996) supra. Briefly, a first hybridization array of probes is used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild type gene and the other complementary to the mutant gene.

In yet another embodiment, any of a variety of sequencing reactions known in the art can be used to directly sequence the 58128 gene in a biological sample and detect mutations by comparing the sequence of the 58128 in the biological sample with the corresponding wild type (control) sequence. Examples of sequencing reactions include those based on techniques developed by Maxam and Gilbert (1977) Proc. Natl. Acad Sci. USA 74:560) and Sanger (1977) Proc. Natl. Acad Sci. USA 74:5463). Furthermore, any of a variety of automated sequencing procedures can be utilized to perform the diagnostic assays (Naeve, C. W. (1995) Biotechniques 19:448-53), including, e.g., sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 58128 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild type 58128 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNAse and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 58128 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 58128 sequence, e.g., a wild type 58128 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility are used to identify mutations in 58128 genes. For example, single strand conformation polymorphism (SSCP) can be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 58128 nucleic acids are denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the migration of mutant or wild type fragments in polyacrylamide gels containing a gradient of denaturant is assayed by denaturing gradient gel electrophoresis (DGGE) (Myers et al. (1985) Nature 313:495). When DGGE is used as the method of analysis, the DNA is modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). In certain embodiments, amplification can also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 58128 modulator (e.g., an agonist, antagonist, peptidomimetic, protein, peptide, nucleic acid, or small molecule) to effectively treat a body weight disorder.

C. Monitoring of Effects During Clinical Trials

The invention further provides methods for determining the effectiveness of a 58128 modulator (e.g., a 58128 modulator identified herein) in treating a body weight disorder in a subject. For example, the effectiveness of a 58128 modulator in increasing 58128 gene expression, protein levels, or in upregulating 58128 activity, can be monitored in clinical trials of subjects exhibiting decreased 58128 gene expression, protein levels, or downregulated 58128 activity. Alternatively, the effectiveness of a 58128 modulator in decreasing 58128 gene expression, protein levels, or in downregulating 58128 activity, can be monitored in clinical trials of subjects exhibiting increased 58128 gene expression, protein levels, or 58128 activity. In such clinical trials, the expression or activity of a 58128 gene, and preferably, other genes that have been implicated in, for example, a body weight disorder can be used as a “read out” or marker of the phenotype of a particular cell.

For example, and not by way of limitation, genes, including 58128, that are modulated in cells by treatment with an agent which modulates 58128 activity (e.g., identified in a screening assay as described herein) can be identified. Thus, to study the effect of agents which modulate 58128 activity on subjects suffering from a body weight disorder participating in, for example, a clinical trial, cells can be isolated and RNA prepared and analyzed for the levels of expression of 58128 and other genes implicated in the body weight disorder. The levels of gene expression (e.g., a gene expression pattern) can be quantified by Northern blot analysis or RT-PCR, as described herein, or alternatively by measuring the amount of protein produced, by any one of the methods described herein, or by measuring the levels of activity of 58128 proteins or other proteins. In this way, the gene expression pattern can serve as a marker which is indicative of the physiological response of the cells to the agent which modulates 58128 activity. This response state may be determined prior to and at various points during treatment of the individual with the agent which modulates 58128 activity.

In a preferred embodiment, the invention provides a method for monitoring the effectiveness of treatment of a subject with an agent which modulates 58128 activity (e.g., an agonist, antagonist, peptidomimetic, protein, peptide, nucleic acid, or small molecule identified by the screening assays described herein) which includes the steps of (i) obtaining a pre-administration sample from a subject prior to administration of the agent; (ii) detecting the level of expression of a 58128 protein, mRNA, or genomic DNA in the pre-administration sample; (iii) obtaining one or more post-administration samples from the subject; (iv) detecting the level of expression or activity of the 58128 protein, mRNA, or genomic DNA in the post-administration samples; (v) comparing the level of expression or activity of the 58128 protein, mRNA, or genomic DNA in the pre-administration sample with the 58128 protein, mRNA, or genomic DNA in the post administration sample or samples; and (vi) altering the administration of the agent to the subject accordingly. For example, increased administration of the agent may be desirable to increase the expression or activity of 58128 to higher levels than detected, i.e., to increase the effectiveness of the agent. Alternatively, decreased administration of the agent may be desirable to decrease expression or activity of 58128 to lower levels than detected, i.e. to decrease the effectiveness of the agent. According to such an embodiment, 58128 expression or activity may be used as an indicator of the effectiveness of an agent, even in the absence of an observable phenotypic response.

Methods of Treatment of Subjects Suffering From Body Weight Disorders

The invention provides for both prophylactic and therapeutic methods of treating a subject, e.g., a human, at risk of (or susceptible to) a body weight disorder such as obesity, overweight, anorexia, cachexia, insulin resistance, or diabetes. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) developing the disease or disorder. As used herein, a “therapeutic agent” includes, but is not limited to, small molecules, peptides, polypeptides, antibodies, ribozymes, and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the 58128 molecules of the invention or 58128 modulators according to that individual's drug response genotype. Pharmacogenomics allows a clinician or physician to target prophylactic or therapeutic treatments to patients who will most benefit from the treatment and to identify patients who will experience toxic drug-related side effects.

A. Prophylactic Methods

In one aspect, the invention provides a method for preventing in a subject, a body weight disorder by administering to the subject an agent which modulates 58128 expression or 58128 activity. Subjects at risk for a body weight disorder can be identified by, for example, any, or a combination, of the diagnostic or prognostic assays described herein. Administration of a prophylactic agent can occur prior to the manifestation of symptoms characteristic ofaberrant 58128 expression or activity, such that the body weight disorder is prevented or, alternatively, delayed in its progression. Depending on the type of 58128 aberrant expression or activity, for example, a 58128 molecule, 58128 agonist or 58128 antagonist agent can be used for treating the subject. The appropriate agent can be determined based on screening assays described herein.

B. Therapeutic Methods

Another aspect of the invention pertains to methods for treating a subject suffering from a body weight disorder. These methods involve administering to a subject an agent which modulates 58128 expression or activity (e.g., an agent identified by a screening assay described herein), or a combination of such agents. In another embodiment, the method involves administering to a subject a 58128 protein or nucleic acid molecule as therapy to compensate for reduced, aberrant, or unwanted 58128 expression or activity.

Stimulation of 58128 activity is desirable in situations in which 58128 is abnormally downregulated and/or in which increased 58128 activity is likely to have a beneficial effect, thereby ameliorating a body weight disorder such as anorexia or cachexia in a subject. Likewise, inhibition of 58128 activity is desirable in situations in which 58128 is abnormally upregulated and/or in which decreased 58128 activity is likely to have a beneficial effect, thereby ameliorating a body weight disorder such as obesity, overweight, or diabetes in a subject.

The agents which modulate 58128 activity can be administered to a subject using pharmaceutical compositions suitable for such administration. Such compositions typically comprise the agent (e.g., a peptide, protein, antibody, or a fragment thereof, peptidomimetic, small molecule, ribozyme, or 58128 antisense molecule) and a pharmaceutically acceptable carrier. As used herein, a “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral (e.g., intravenous, intradermal, subcutaneous, intranasal, or intramuscular), oral, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be achieved by including in the composition an agent which delays absorption, for example, aluminum monostearate and gelatin.

Sterile injectable solutions can be prepared by incorporating the agent that modulates 58128 activity (e.g., a peptide, protein or antibody, or fragment thereof, peptidomimetic, small molecule, ribozyme, or 58128 antisense molecule) in the required amount in an appropriate solvent with one or a combination of the ingredients enumerated above, as required, followed by filtered sterilization. Generally, dispersions are prepared by incorporating the active compound into a sterile vehicle which contains a basic dispersion medium and other required ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with one or more excipients and administered in the form of a liquid, tablet, troche (e.g., a lozenge), or capsule. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration includes transmucosal or transdernal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 58128 activity can also be prepared in the form of suppositories (e.g., with conventional suppository bases such as cocoa butter and other glycerides) or retention enemas for rectal delivery.

In one embodiment, the agents that modulate 58128 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation. Liposomal suspensions (including liposomes targeted to virus-infected cells with monoclonal antibodies to the viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit-form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and are directly dependent on the unique characteristics of the agent that modulates 58128 activity and the particular therapeutic effect to be achieved, as well as the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in the formulation of a range of dosage for use in humans. The dosage of such 58128 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (ie., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including, but not limited to, the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The invention encompasses agents which modulate 58128 gene expression or 58128 protein activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses can be determined using any of the assays described herein. When one or more of these small molecules is to be administered to an animal (e.g., a human) in order to modulate expression or activity of a polypeptide or nucleic acid of the invention, a physician, veterinarian, or researcher may, for example, initially prescribe a relatively low dose, and subsequently increasing the dose until an appropriate response is obtained. In addition, it is understood that the specific dose level for any particular animal subject will depend upon a variety of factors including the activity of the specific compound to be administered, the age, body weight, general health, gender, and diet of the subject, the time of administration, the route of administration, the rate of excretion, any drug combination, and the degree of expression or activity to be modulated.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) csn be considered. Individual differences in the metabolism of therapeutics can lead to severe toxicity or therapeutic failure by altering the relation between dose and blood concentration of a pharmacologically active drug. Thus, a physician or clinician may consider applying knowledge obtained in relevant pharmacogenomics studies in determining whether to administer an agent which modulates 58128 activity to a subject, as well as tailoring the dosage and/or therapeutic regimen of treatment with an agent which modulates 58128 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor that alters the way drugs act on the body (altered drug action) and genetic conditions transmitted as a single factor that alters the way the body acts on drugs (altered drug metabolism). These pharmacogenetic conditions can occur either as rare genetic defects or as naturally occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, although, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 58128 protein of the invention), all common variants of that gene can be fairly easily identified in the population and it can then be determined whether having a particular variant of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show an exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience an exaggerated drug response and side effects when they receive standard doses. If a metabolite is the active therapeutic moiety, PM show no therapeutic response, as demonstrated by the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified as a result of CYP2D6 gene amplification.

Alternatively, a method termed the “gene expression profiling” can be utilized to identify genes that predict drug response. For example, the gene expression of an animal dosed with a drug (e.g., a 58128 molecule or 58128 modulator of the invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosage determination or drug selection, can reduce or prevent adverse reactions or therapeutic failure and, thus, enhance therapeutic or prophylactic efficiency when treating a subject suffering from a body weight disorder with an agent which modulates 58128 activity.

Isolated Nucleic Acid Molecules Used In the Methods of the Invention

The methods of the invention (e.g., the screening assays described herein) include the use of58128 nucleic acid molecules. The cDNA sequence of the isolated human 58128 gene and the predicted amino acid sequence of the human 58128 polypeptide are shown in SEQ ID NOs:1 and 2, respectively. The sequence of the open reading frame of human 58128 is shown in SEQ ID NO:3. The cDNA sequence of the isolated murine 58128 gene and the predicted amino acid sequence of the mouse 58128 polypeptide are shown in SEQ ID NOs:4 and 5, respectively. When aligned using the ALIGN program (version 2.0; see Myers and Miller (1989) CABIOS), the mouse and human 58128 nucleotide sequences are about 78% identical. When aligned using the ALIGN program (version 2.0; Myers and Miller supra), the mouse and human 58128 amino acid sequences are about 81% identical.

The methods of the invention include the use of isolated nucleic acid molecules that encode 58128 proteins, or biologically active portions thereof, as well as nucleic acid fragments sufficient for use as hybridization probes, to identify 58128 encoding nucleic acid molecules (e.g., 58128 mRNA) and fragments for use as PCR primers for the amplification or mutation of 58128 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the invention, e.g., a nucleic acid molecule having the nucleotide.sequence of SEQ ID NO:1, 3, or 4, or a portion thereof, can be isolated using standard molecular biology techniques and the sequence information provided herein. Using all or portion of the nucleic acid sequence of SEQ ID NO:1, 3, or 4 as a hybridization probe, 58128 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3 or 4 can be isolated by polymerase chain reaction (PCR) using synthetic oligonucleotide primers designed based upon the sequence of SEQ ID NO:1, 3, or 4.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 58128 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, or 4, a complement of the nucleotide sequence shown in SEQ ID NO:1, 3, or 4, or a portion of any of these nucleotide sequences. A nucleic acid molecule which is complementary to the nucleotide sequence shown in SEQ ID NO:1, 3, or 4, is one which is sufficiently complementary to the nucleotide sequence shown in SEQ ID NO:1, 3, or 4 such that it can hybridize to the nucleotide sequence shown in SEQ ID NO:1, 3, or 4 thereby forming a stable duplex.

In still another preferred embodiment, an isolated nucleic acid molecule used in the methods of the invention comprises a nucleotide sequence which is at least about 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the nucleotide sequence shown in SEQ ID NO:1, 3, or 4, or a portion of any of this nucleotide sequence.

Moreover, the nucleic acid molecules used in the methods of the invention can comprise only a portion of the nucleic acid sequence of SEQ ID NO:1, 3, or 4, for example, a fragment which can be used as a probe or primer or a fragment encoding a portion of a 58128 protein, e.g., a biologically active portion of a 58128 protein. The probe or primer typically comprises a substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, or 4 or an anti-sense sequence of SEQ ID NO:1, 3, or 4, or of a naturally occurring allelic variant or mutant of SEQ ID NO:1, 3, or 4. In one embodiment, a nucleic acid molecule used in the methods of the invention comprises a nucleotide sequence which is greater than 50, 50-100, 100-200, 200-300, 300-400, 400-500, 500-600, 600-700, 700-800, 800-900, 900-1000, or more nucleotides in length and hybridizes under stringent hybridization conditions to a nucleic acid molecule of SEQ ID NO:1 , 3, or 4.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× or 6× sodium chloride/sodium citrate (SSC), at about 65-70° C. (or hybridization in 4×SSC plus 50% formamide at about 42-50° C.) followed by one or more washes in 1×SSC, at about 65-70° C. A further preferred, non-limiting example of stringent hybridization conditions includes hybridization at 6×SSC at 45° C., followed by one or more washes in 0.2×SSC, 0.1% SDS at 65° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4× or 6×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the invention. SSPE (1×SSPE is 0.15M NaCl, 10mM NaH₂PO₄, and 1.25mM EDTA, pH 7.4) can be substituted for SSC (1×SSC is 0.15M NaCl and 15mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP, and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 58128 protein, such as by measuring a level of a 58128 encoding nucleic acid in a sample of cells from a subject e.g., detecting 58128 mRNA levels or determining whether a genomic 58128 gene has been mutated or deleted.

The methods of the invention further encompass the use of nucleic acid molecules that differ from the nucleotide sequence shown in SEQ ID NO:1, 3, or 4 due to degeneracy of the genetic code and thus encode the same 58128 proteins as those encoded by the nucleotide sequence shown in SEQ ID NO:1, 3, or 4 In another embodiment, an isolated nucleic acid molecule included in the methods of the invention has a nucleotide sequence encoding a protein having an amino acid sequence shown in SEQ ID NO:2 or 5.

The methods of the invention further include the use of allelic variants of human 58128, e.g., functional and non-functional allelic variants. Functional allelic variants are naturally occurring amino acid sequence variants of the human 58128 protein that maintain a 58128 activity. Functional allelic variants will typically contain only conservative substitution of one or more amino acids of SEQ ID NO:2 or 5, or substitution, deletion or insertion of non-critical residues in non-critical regions of the protein. Non-functional allelic variants are naturally occurring amino acid sequence variants of the human 58128 protein that do not have a 58128 activity. Non-functional allelic variants typically contain a non-conservative substitution, deletion, or insertion or premature truncation of the amino acid sequence of SEQ ID NO:2 or 5, or a substitution, insertion or deletion in critical residues or critical regions of the protein.

The methods of the invention can further use non-human orthologues of the human 58128 protein. Orthologues of the human 58128 protein are proteins that are isolated from non-human organisms and possess the same 58128 activity.

The methods of the invention further include the use of nucleic acid molecules comprising the nucleotide sequence of SEQ ID NO:1, 3, or 4, or a portion thereof, in which a mutation has been introduced. The mutation may lead to amino acid substitutions at “non-essential” amino acid residues or at “essential” amino acid residues. A “non-essential” amino acid residue is a residue that can be altered from the wild type sequence of 58128 (e.g., the sequence of SEQ ID NO:2 or 5) without altering the biological activity, whereas an “essential” amino acid residue is required for biological activity. For example, amino acid residues that are conserved among the 58128 proteins of the invention and other members of the bigenic amine-like receptor subfamily of GPCRs are not likely to be amenable to alteration.

Mutations can be introduced into SEQ ID NO:1, 3, or 4 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 58128 protein is preferably replaced with another amino acid residue from the same side chain family. Alternatively, in another embodiment, mutations can be introduced randomly along all or part of a 58128 coding sequence, such as by saturation mutagenesis, and the resultant mutants can be screened for 58128 biological activity to identify mutants that retain activity. Following mutagenesis of SEQ ID NO:1, 3, or 4, the encoded protein can be expressed recombinantly and the activity of the protein can be determined using an assay described herein.

Another aspect of the invention pertains to the use of isolated nucleic acid molecules which are antisense to the nucleotide sequence of SEQ ID NO:1, 3, or 4. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 58128 coding strand, or to only a portion thereof. In one embodiment, an antisense nucleic acid molecule is antisense to a “coding region” of the coding strand of a nucleotide sequence encoding a 58128. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 58128. The term “noncoding region” refers to 5′ and 3′ sequences which flank the coding region that are not translated into amino acids (also referred to as 5′ and 3′ untranslated regions).

Given the coding strand sequences encoding 58128 disclosed herein, antisense nucleic acids of the invention can be designed according to the rules of Watson and Crick base pairing. The antisense nucleic acid molecule can be complementary to the entire coding region of 58128 mRNA, but more preferably is an oligonucleotide which is antisense to only a portion of the coding or noncoding region of 58128 mRNA. For example, the antisense oligonucleotide can be complementary to the region surrounding the translation start site of 58128 mRNA. An antisense oligonucleotide can be, for example, about 5, 10, 15, 20, 25, 30, 35,40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid is in an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a 58128 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave 58128 mRNA transcripts to thereby inhibit translation of 58128 mRNA. A ribozyme having specificity for a 58128 encoding nucleic acid can be designed based upon the nucleotide sequence of a 58128 cDNA disclosed herein (i.e., SEQ IDNO:1, 3, or 4). For example, a derivative of a Tetrahymena L-19 IVS RNA can be constructed in which the nucleotide sequence of the active site is complementary to the nucleotide sequence to be cleaved in a 58128 encoding mRNA. See, e.g., Cech et al. U.S. Pat. No.4,987,071; and Cech et al. U.S. Pat. No. 5,116,742. Alternatively, 58128 mRNA can be used to select a catalytic RNA having a specific ribonuclease activity from a pool of RNA molecules. See, e.g., Bartel, D. and Szostak, J. W. (1993) Science 261:1411-1418.

Alternatively, 58128 gene expression can be inhibited by targeting nucleotide sequences complementary to the regulatory region of the 58128 (e.g., the 58128 promoter and/or enhancers) to form triple helical structures that prevent transcription of the 58128 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioessays 14(12):807-15.

In yet another embodiment, the 58128 nucleic acid molecules used in the methods of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup and Nielsen (1996) Bioorg. Med. Chem. 4:5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. and Nielsen (1996) supra and Perry-O'Keefe et al. (1996) Proc. Natl. Acad. Sci. USA 93:14670-675.

PNAs of 58128 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 58128 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of 58128 can be modified, (e.g., to enhance their stability or cellular uptake), by attaching lipophilic or other helper groups to the PNA, by the formation of PNA-DNA chimeras, or by the use of liposomes or other techniques of drug delivery known in the art. For example, PNA-DNA chimeras of 58128 nucleic acid molecules can be generated which may combine the advantageous properties of a PNA and DNA. Such chimeras allow DNA recognition enzymes, (e.g., RNAse H and DNA polyrnerases), to interact with the DNA portion while the PNA portion provides high binding affinity and specificity. PNA-DNA chimeras can be linked using linkers of appropriate lengths selected on the basis of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17: 5973-88). PNA monomers are then coupled in a stepwise manner to produce a chimeric molecule with a 5′ PNA segment and a 3′ DNA segment (Finn et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre etal. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (See, e.g., Krol et al. (1988) Biotechniques 6:958-976) or intercalating agents. (See, e.g., Zon (1988) Pharm. Res. 5:539-549). To this end, the oligonucleotide may be conjugated to another molecule (e.g., a peptide, hybridization triggered cross-linking agent, transport agent, or hybridization-triggered cleavage agent).

Recombinant Expression Vectors And Host Cells Used In the Methods of the Invention

The methods of the invention (e.g., the screening assays described herein) include the use of vectors, preferably expression vectors, containing a nucleic acid encoding a 58128 protein, or a portion or fragment thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” are used interchangeably herein, given that the plasmid is the most commonly used form of vector. However, the invention is intended to include other forms of expression vectors, such as viral vectors (e.g., replication defective retroviruses, adenoviruses and adeno-associated viruses), which serve equivalent functions.

The recombinant expression vectors used in the methods of the invention comprise a nucleic acid of the invention in a form suitable for expression of the nucleic acid in a host cell. As a consequence, recombinant expression vectors include one or more regulatory sequences which is selected on the basis of the host cells to be used for expression and which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 58128 proteins, mutant forms of 58128 proteins, fusion proteins, and the like).

The recombinant expression vectors to be used in the methods of the invention can be designed for expression of 58128 proteins in prokaryotic or eukaryotic cells. For example, 58128 proteins can be expressed in bacterial cells (such as E. coli), insect cells (using baculovirus expression vectors), yeast cells, or mammalian cells (such as Chinese hamster ovary (CHO) cells or SV40 transformed African green monkey kidney (COS-7) cells). Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Amersham Pharmacia Biotech, Inc., Piscataway, N.J.) which fuse glutathione S-transferase (GST), maltose E binding protein, or protein A, respectively, to the target recombinant protein.

Purified fusion proteins can be utilized in 58128 activity assays, (e.g., direct assays or competitive assays described herein), or to generate antibodies specific for 58128 proteins. In a preferred embodiment, a 58128 fusion protein expressed in a retroviral expression vector of the invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells, see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 58128 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 58128 nucleic acid molecule of the invention is introduced, e.g., a 58128 nucleic acid molecule within a recombinant expression vector or a 58128 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 58128 protein can be expressed in bacterial cells such as E. coli, insect cells, yeast or mammalian cells (such as CHO or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 58128 protein. Accordingly, the invention further provides methods for producing a 58128 protein using the host cells of the invention. In one embodiment, the method comprises culturing the host cell of the invention (into which a recombinant expression vector encoding a 58128 protein has been introduced) in a suitable medium such that a 58128 protein is produced. In another embodiment, the method further comprises isolating a 58128 protein from the medium or the host cell.

Isolated 58128 Proteins And Anti-58128 Antibodies Used In the Methods of the Invention

The methods of the invention (e.g., the screening assays described herein) include the use of isolated 58128 proteins, and biologically active portions thereof, as well as polypeptide fragments suitable for use as immunogens to raise anti-58128 antibodies. In one embodiment, naturally occurring 58128 proteins can be isolated from cells or tissue sources by an appropriate purification scheme using standard protein purification techniques. In another embodiment, 58128 proteins are produced by recombinant DNA techniques. Alternative to recombinant expression, a 58128 protein or polypeptide can be synthesized chemically using standard peptide synthesis techniques.

As used herein, a “biologically active portion” of a 58128 protein includes a fragment of a 58128 protein having a 58128 activity. Biologically active portions of a 58128 protein include peptides comprising amino acid sequences sufficiently identical to or derived from the amino acid sequence of the 58128 protein, e.g., the amino acid sequence shown in SEQ ID NO:2 or 5, which include fewer amino acids than the full length 58128 proteins, and exhibit at least one activity of a 58128 protein. Typically, biologically active portions comprise a domain or motif with at least one activity of the 58128 protein. A biologically active portion of a 58128 protein can be a polypeptide which is, for example, 25, 50, 75, 100, 125, 150, 175, 200, 250, 300 or more amino acids in length. Biologically active portions of a 58128 protein can be used as targets for developing agents which modulate a 58128 activity.

In a preferred embodiment, the 58128 protein used in the methods of the invention has an amino acid sequence shown in SEQ ID NO:2 or 5. In other embodiments, the 58128 protein is substantially identical to SEQ ID NO:2 or 5, and retains the functional activity of the protein of SEQ ID NO:2 or 5, yet differs in amino acid sequence due to natural allelic variation or mutagenesis. Accordingly, in another embodiment, the 58128 protein used in the methods of the invention is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2 or 5.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 58128 amino acid sequence of SEQ ID NO:2 having 306 amino acid residues, at least 92, preferably at least 123, more preferably at least 153, even more preferably at least 184, and even more preferably at least 214, 245, 276 or more amino acid residues are aligned; when aligning a second sequence to the 58128 amino acid sequence of SEQ ID NO:5 having 276 amino acid residues, at least 83, preferably at least 111, more preferably at least 132, even more preferably at least 166, and even more preferably at least 194, 222, 249 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention also use 58128 chimeric or fusion proteins. As used herein, a 58128 “chimeric protein” or “fusion protein” comprises a 58128 polypeptide operatively linked to a non-58128 polypeptide. A “58128 polypeptide” refers to a polypeptide having an amino acid sequence corresponding to a 58128 molecule, whereas a “non-58128 polypeptide” refers to a polypeptide having an amino acid sequence corresponding to a protein which is not substantially homologous to the 58128 protein, e.g., a protein which is different from the 58128 protein and which is derived from the same or a different organism. Within a 58128 fusion protein the 58128 polypeptide can correspond to all or a portion of a 58128 protein (e.g., the amino acid sequence shown in SEQ ID NO:2 or 5). In a preferred embodiment, a 58128 fusion protein comprises at least one biologically active portion of a 58128 protein. In another preferred embodiment, a 58128 fusion protein comprises at least two biologically active portions of a 58128 protein. Within the fusion protein, the term “operatively linked” is intended to indicate that the 58128 polypeptide and the non-58128 polypeptide are fused in-frame to each other. The non-58128 polypeptide can be fused to the N-terminus or C-terminus of the 58128 polypeptide.

For example, in one embodiment, the fusion protein is a GST-58128 fusion protein in which the 58128 sequence is fused to the C-terminus of the GST sequence. Such a fusion protein can facilitate the purification of recombinant 58128.

In another embodiment, this fusion protein is a 58128 protein containing a heterologous signal sequence at its N-terminus. In certain host cells (e.g., mammalian host cells), expression and/or secretion of 58128 can be increased through use of a heterologous signal sequence.

The 58128 fusion proteins used in the methods of the invention can be incorporated into pharmaceutical compositions and administered to a subject in vivo. The 58128 fusion proteins can be used to alter the bioavailability of a 58128 substrate. 58128 fusion proteins may be useful therapeutically for the treatment of disorders caused by, for example, (i) aberrant modification or mutation of a gene encoding a 58128 protein; (ii) mis-regulation of the 58128 gene; and (iii) aberrant post-translational modification of a 58128 protein.

Moreover, the 58128-fusion proteins used in the methods of the invention can be used as immunogens to produce anti-58128 antibodies in a subject, to purify 58128 ligands and in screening assays to identify molecules which inhibit the interaction of 58128 with a 58128 substrate.

Preferably, a 58128 chimeric or fusion protein used in the methods of the invention is produced by standard recombinant DNA techniques. For example, DNA fragments encoding different polypeptide sequences can be ligated in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers to produce complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, expression vectors which encode a fusion moiety (e.g., a GST polypeptide) are commercially available. A 58128 encoding nucleic acid can be cloned into such an expression vector such that the fusion moiety is linked in-frame to the 58128 protein.

The invention also pertains to the use of variants of the 58128 proteins which function as either 58128 agonists (mimetics) or as 58128 antagonists. Variants of the 58128 proteins can be generated by mutagenesis, e.g., discrete point mutation or truncation of a 58128 protein. An agonist of the 58128 proteins can retain substantially the same, or a subset, of the biological activities of the naturally occurring form of a 58128 protein. An antagonist of a 58128 protein can inhibit one or more of the activities of the naturally occurring form of the 58128 protein by, for example, competitively modulating a 58128 mediated activity of a 58128 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 58128 protein.

In one embodiment, variants of a 58128 protein which function as either 58128 agonists (mimetics) or as 58128 antagonists can be identified by screening combinatorial libraries of mutants, e.g., truncation mutants, of a 58128 protein for 58128 protein agonist or antagonist activity. In one embodiment, a variegated library of 58128 variants is generated by combinatorial mutagenesis at the nucleic acid level and is encoded by a variegated gene library. A variegated library of 58128 variants can be produced by, for example, enzymatically ligating a mixture of synthetic oligonucleotides into gene sequences such that a degenerate set of potential 58128 sequences is expressible as individual polypeptides, or alternatively, as a set of larger fusion proteins (e.g., for phage display) containing the set of 58128 sequences therein. There are a variety of methods which can be used to produce libraries of potential 58128 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 58128 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 58128 protein coding sequence can be used to generate a variegated population of 58128 fragments for screening and subsequent selection of variants of a 58128 protein. In one embodiment, a library of coding sequence fragments can be generated by treating a double stranded PCR fragment of a 58128 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 58128 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 58128 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 58128 variants (Arkin and Youvan (1992) Proc. Natl. Acad. Sci. USA 89:7811-7815; Delagrave et al. (1993) Prot. Eng. 6(3):327-331).

The methods of the invention further include the use of anti-58128 antibodies. An isolated 58128 protein, or a portion or fragment thereof, can be used as an immunogen to generate antibodies that bind 58128 using standard techniques for polyclonal and monoclonal antibody preparation. A full-length 58128 protein can be used or, alternatively, antigenic peptide fragments of 58128 can be used as immunogens. The antigenic peptide of 58128 comprises at least 8 amino acid residues of the amino acid sequence shown in SEQ ID NO:2 or 5 and encompasses an epitope of 58128 such that an antibody raised against the peptide forms a specific immune complex with the 58128 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 58128 that are located on the surface of the protein, e.g., hydrophilic regions, as well as regions with high antigenicity.

A 58128 immunogen is typically used to prepare antibodies by immunizing a suitable subject, (e.g., rabbit, goat, mouse, or other mammal) with the immunogen. An appropriate immunogenic preparation can contain, for example, recombinantly expressed 58128 protein or a chemically synthesized 58128 polypeptide. The preparation can further include an adjuvant, such as Freund's complete or incomplete adjuvant, or similar immunostimulatory agent. Immunization of a suitable subject with an immunogenic 58128 preparation induces a polyclonal anti-58128 antibody response.

The term “antibody” as used herein refers to immunoglobulin molecules and immunologically active portions of immunoglobulin molecules, i.e., molecules that contain an antigen binding site which specifically binds (e.g., immunoreacts with) an antigen, such as a 58128 polypeptide. Examples of immunologically active portions of immunoglobulin molecules include single chain FV (scFV) and double chain FV (dcFV) fragments, Fab and F(ab′)₂ fragments which can be generated by treating the antibody with an enzyme such as papain or pepsin, respectively. The invention provides polyclonal and monoclonal antibodies that bind 58128 molecules. The term “monoclonal antibody” or “monoclonal antibody composition”, as used herein, refers to a population of antibody molecules that contain only one species of an antigen binding site capable of immunoreacting with a particular epitope of 58128. A monoclonal antibody composition thus typically displays a single binding affinity for a particular 58128 protein with which it immunoreacts.

Polyclonal anti-58128 antibodies can be prepared as described above by immunizing a suitable subject with a 58128 immunogen. The anti-58128 antibody titer in the immunized subject can be monitored over time by standard techniques, such as with an enzyme linked immunosorbent assay (ELISA) using immobilized 58128. If desired, the antibody molecules directed against 58128 can be isolated from the mammal (e.g., from the blood) and further purified by well known techniques, such as protein A chromatography to obtain the IgG fraction. At an appropriate time after immunization, e.g., when the anti-58128 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med 54:387-402; Gefter, M. L. et al. (1977) Somat. Cell Genet. 3:231-36). Briefly, an immortal cell line (typically a myeloma) is fused to lymphocytes (typically splenocytes) from a mammal immunized with a 58128 immunogen as described above, and the culture supernatants of the resulting hybridoma cells are screened to identify a hybridoma producing a monoclonal antibody that binds 58128.

Any of the many well known protocols used for fusing lymphocytes and immortalized cell lines can be applied for the purpose of generating an anti-58128 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 58128, e.g., using a standard ELISA assay.

Alternative to preparing monoclonal antibody-secreting hybridomas, a monoclonal anti-58128 antibody can be identified and isolated by screening a recombinant combinatorial immunoglobulin library (e.g., an antibody phage display library) with 58128 to thereby isolate immunoglobulin library members that bind 58128. Kits for generating and screening phage display libraries are commercially available (e.g., Recombinant Phage Antibody System (Amersham Pharmacia Biotech, Inc.); and the Stratagene SurfZAP™ Phage Display Kit). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, chimeric, humanized, and completely human antibodies are also within the scope of the invention. Chimeric, humanized, but most preferably, completely human antibodies are desirable for applications which include repeated administration, e.g., therapeutic treatment of human patients, and some diagnostic applications.

Chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

A humanized or complementarity determining region (CDR)-grafted antibody will have at least one or two, but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 58128 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison (1985) Science 229:1202-1207, by Oi et al. (1986) BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 58128 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. (1986) Nature 321:552-525; Verhoeyan et al. (1988) Science 239:1534; Beidler et al. (1988) J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (U.S. Pat. No. 5,225,539, the contents of which is expressly incorporated by reference).

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-58128 antibody can be a single chain antibody. A single-chain antibody (scFV) can be engineered as described in, for example, Colcher et al. (1999) Ann. N Y Acad. Sci. 880:263-80; and Reiter (1996) Clin. Cancer Res. 2:245-52. The single chain antibody can be dimerized or multimerized to generate multivalent antibodies having specificities for different epitopes of the same target 58128 protein.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp.303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

Alternatively, an antibody can be conjugated to a second antibody to form an antibody heteroconjugate as described by Segal in U.S. Pat. No. 4,676,980.

An anti-58128 antibody (e.g., monoclonal antibody) can be used to isolate 58128 by standard techniques, such as affinity chromatography or immunoprecipitation. Moreover, an anti-58128 antibody can be used to detect 58128 protein (e.g., in a cellular lysate or cell supernatant) in order to evaluate the abundance and pattern of expression of the protein. Anti-58128 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In preferred embodiments, an antibody can be made by immunizing with a purified 58128 antigen, or a fragment thereof, e.g., a fragment described herein, a membrane associated antigen, tissues, e.g., crude tissue preparations, whole cells, preferably living cells, lysed cells, or cell fractions, e.g., membrane fractions.

Antibodies which bind only a native 58128 protein, only denatured or otherwise non-native 58128 protein, or which bind both, are within the invention. Antibodies with linear or conformational epitopes are within the invention. Conformational epitopes sometimes can be identified by identifying antibodies which bind to native but not denatured 58128 protein.

This invention is further illustrated by the following examples which should not be construed as limiting. The contents of all references, patents and published patent applications cited throughout this application, as well as the Sequence Listing, are incorporated herein by reference.

### EXAMPLES

### Example 1

### Gene Expression Analysis

Total RNA was prepared from various human tissues by a single step extraction method using RNA STAT-60 according to the manufacturer's instructions (TelTest, Inc). Each RNA preparation was treated with DNase I (Ambion) at 37° C. for 1 hour. DNAse I treatment was determined to be complete if the sample required at least 38 PCR amplification cycles to reach a threshold level of fluorescence using β2-microglobulin as an internal amplicon reference. The integrity of the RNA samples following DNAse I treatment was confirmed by checking the 18s/28s ratios using an Agilent 2100 Bioanalyzer (Agilent Technologies, Inc.). After phenol extraction cDNA was prepared from the sample using the SUPERSCRIPT™ Choice System following the manufacturer's instructions (GibcoBRL). A negative control of RNA without reverse transcriptase was mock reverse transcribed for each RNA sample. Human 58128 expression was measured by TaqMan 4 quantitative PCR (Perkin Elmer Applied Biosystems) in cDNA prepared from a variety of normal and diseased (e.g., cancerous) human tissues.

Probes were designed by PrimerExpress software (PE Applied Biosystems) based on the sequence of the human 58128 gene. Each human 58128 gene probe was labeled using FAM (6-carboxyfluorescein), and the Beta 2-microglobulin reference probe was labeled with a different fluorescent dye, VIC. The differential labeling of the target gene and internal reference gene thus enabled measurement in same well. Forward and reverse primers and the probes for both β2-microglobulin and target gene were added to the TaqMan® Universal PCR Master Mix (PE Applied Biosystems). Although the final concentration of primer and probe could vary from experiment to experiment, each was internally consistent within a given experiment. A typical experiment contained 100nM of forward and reverse primers plus 200nM probe for β2-microglobulin and 900 nM forward and reverse primers plus 250 nM probe for the target gene. TaqMan matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec, followed by 60° C. for 1 min.

The following method was used to quantitatively calculate human 58128 gene expression in the various tissues relative to β2-microglobulin expression in the same tissue. The threshold cycle (Ct) value is defined as the cycle at which a statistically significant increase in fluorescence is detected. A lower Ct value is indicative of a higher mRNA concentration. The Ct value of the human 58128 gene is normalized by subtracting the Ct value of the β2-microglobulin gene to obtain a _{Δ}Ct value using the following formula: _{Δ}Ct=Ct_{human 58128}−Ct_{β2}-microglobulin. Expression is then calibrated against a cDNA sample showing a comparatively low level of expression of the human 58128 gene. The _{Δ}Ct value for the calibrator sample is then subtracted from _{Δ}Ct for each tissue sample according to the following formula: _{ΔΔ}Ct=_{Δ}Ct-ₛₐₘₚₗₑ−_{Δ}Ct-_{calbrator}. Relative expression is then calculated using the arithmetic formula given by 2^{-ΔΔCt}. The results indicate significant 58128 expression in hypothalamus.

The distribution of 58128 mRNA in mouse brain was examined by in situ hybridization. Mouse brain was frozen with powdered dry ice, and cryostat sections were cut at 10 μm thickness through hypothalamus region, mounted on Superfrost Plus microscope slides (Erie Scientific Co.) and stored at −80° until needed.

Prior to analysis, mouse brain sections were air dried for 20 minutes and then incubated with ice cold 4% PFA (paraformaldehyde)/1×PBS for 10 minutes. The slides were then washed with 1×PBS twice (5 minutes each time), incubated with 0.25% acetic anhydride/1M triethanolamine for 10 minutes, washed with PBS for 5 minutes and dehydrated with 70%, 80%, 95% and 100% ethanol (1 minute each). Sections were incubated with chloroform for 5 minutes, rehydrated with 100% and 95% ethanol, then air dried. Hybridizations were performed with ³⁵S-radiolabeled (5×10⁷ cpm/ml) cRNA probes in the presence of 50% formamide, 10% dextran sulfate, 1× Denhardt's solution, 600 mM NaCl, 10 mM DTT, 0.25% SDS and 100 μg/ml RNAse A in TNE at 37° C. for 30 minutes, washed in TNE for 10 minutes, ,incubated once in 2×SSC at 65° for 30 minutes, once in 0.2×SSC at 70° for 30 minutes, 0.2×SSC at 70° for 30 minutes and dehydrated with 50%, 70%, 80%, 95% and 100% ethanol. Localization of mRNA transcripts was detected by dipping slides in Kodak NBT-2 photoemulsion and exposing for 14 days at 4° C., followed by development with Dektol (Eastman Kodak Co.). Slides were counterstained with haemotoxylin and eosin and photographed. Controls for the in situ hybridization experiments included the use of a sense probe which showed no signal above background levels.

This analysis revealed that 58128-mRNA is expressed within the ventral/medial hypothalamus, both of which are implicated in control of feeding behavior.

### Example 2

### Isolation of A cDNA Encoding Murine 58128

The cDNA sequence encoding a partial murine 58128 polypeptide is shown in SEQ ID NO:4. The corresponding predicted amino acid sequence of the murine 58128 is depicted in SEQ ID NO:5. A clone encoding murine 58128 was identified as follows. The partial murine 58128 cDNA was cloned from murine genomic DNA by PCR using cross species primers, a pair of human primers that flank the entire coding region of human 58128. Sequencing of the clones so identified led to the identification of a clone sharing 85% identity with human 58128 DNA sequence and encoding a protein, murine 58128 with a high degree of sequence identity to human 58128.

### Example 3

### Signal Transduction Assays

The activity of murine or human 58128 can be measured using any assay suitable for the measurement of the activity of a G protein-coupled receptor. Signal transduction activity of a G protein-coupled receptor can be monitored by measuring intracellular Ca²⁺, cAMP, inosital 1,4,5-trisphophate (IP₃), or 1,2-diacylglycerol (DAG). Assays for the measurement of intracellular Ca²⁺ are described, for example, in Sakurai et al. (EP 480 381). Intracellular IP₃ can be measured using a commercially available assay kit (Amersham Pharmacia Biotech, Inc.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the media of the host cells expressing 58128. Once inside the cell, the fura-2 ester is hydrolyzed by cytosolic esterases to its non-lipophilic form, which prevents the dye from diffusing out of the cell. The non-lipophilic form of fura-2 will fluoresce when bound to free Ca²⁺. The fluorescence can be measured without lysing the cells at an excitation spectrum of 340 nm or 380 nm and at a fluorescence spectrum of 500 nm (see, e.g., Sakurai et al., EP 480 381).

Upon activation of a G protein-coupled receptor, the rise of free cytosolic Ca²⁺ concentrations is preceded by the hydrolysis of phosphatidylinositol 4,5-bisphosphate. Hydrolysis of this phospholipid by phospholipase C yields 1,2-diacylglycerol (DAG), which remains in the membrane, and water-soluble inositol 1,4,5-trisphophate (IP₃). Binding of ligand or agonists will increase the concentration of DAG and IP₃. Thus, signal transduction activity can be measured by monitoring the concentration of these hydrolysis products.

To measure the IP₃ concentrations, ³H-inositol is added to the media of host cells expressing 58128. The ³H-inositol is taken up by the cells and incorporated into IP₃. The resulting inositol triphosphate is separated from the mono- and di-phosphate forms and measured (Sakurai et al., EP 480 381). Altematively, Amersham provides an inositol 1,4,5-triphosphate assay system. This assay system uses tritylated inositol 1,4,5-triphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described, e.g., in Gilman et al. (1970) Proc. Natl. Acad. Sci. USA 67:305-312.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for identifying a compound capable of treating a body weight disorder, comprising assaying the ability of the compound to modulate 58128 polypeptide activity, thereby identifying a compound capable of treating a body weight disorder, wherein the 58128 polypeptide is a polypeptide comprising the amino acid sequence of SEQ ID NO:2.

2. The method of claim, 1 wherein the body weight disorder is selected from the group consisting of obesity, overweight, diabetes, insulin resistance, cachexia, and anorexia.

3. The method of claim 1, wherein the ability of the compound to modulate 58128 polypeptide activity is determined by detecting a 58128 activity of a cell.

4. The method of claim 1, wherein the 58128 polypeptide is encoded by a nucleic acid molecule which is at least 95% identical to SEQ ID NO:1, and wherein the nucleic acid molecule hybridizes to a complement of a nucleic acid molecule consisting of SEQ ID NO:1 in 6×SSC at 45° C., followed by one or more washes in 0.2×SSC, 0.1% SDS at 65° C.

5. The method of claim 1, comprising:
(a) contacting a cell which expresses 58128 with a test compound; and
(b) assaying the ability of the test compound to modulate activity of a 58128 polypeptide, thereby identifying a compound capable of modulating a GPCR activity when a compound is assessed as having the ability to modulate the activity of the 58128 polypeptide.

6. The method of claim 1, comprising:
(a) contacting a polypeptide comprising the amino acid sequence of SEQ ID NO:2; and
(b) assaying the ability of the test compound to modulate the activity of the polypeptide, thereby identifying a compound capable of modulating a GPCR activity.

7. The method of claim 1, wherein the compound or modulator is a small molecule.

8. The method of claim 1, wherein the compound or modulator is an anti-58128 antibody.

9. The method of claim 6, wherein the cell is a brain cell.

10. The method of claim 6, wherein the assessed activity of the 58128 polypeptide comprises determining an activity selected from the group consisting of altering intracellular Ca²⁺ concentration, activating phospholipase C, altering intracellular inositol, 1,4,5-triphophate concentration, altering intracellular 1,2-diacylglycerol concentration, altering cAMP concentration.

11. The method of claim 6, wherein the assessed activity of the 58128 polypeptide comprises determining altered glucose concentration in the cells or determining phosphorylation of proteins in the insulin pathway.


SEQUENCE LISTING (5 sequences)

SEQ ID NO: 1 - DNA, 1041 bp, Human
  5′UTR (1)...(108)
  CDS (109)...(1029)
  3′UTR (1030)...(1041)
tgacaaaattctatctgttcttgttttttgaaggaaaaattcaattgctctgaatatggaaatagatcttgcccagaaaatgaaagatctctgggtgtccgagtggctatgtattcatttatggcaggatccatattcatcacaatatttggcaatcttgccatgataatttccatttcctacttcaagcagcttcacacaccaaccaacttcctcatcctctccatggccatcactgatttcctcctgggattcaccatcatgccatatagtatgatcagatcggtggagaactgctggtattttgggcttacattttgcaagatttattatagttttgacctgatgcttagcataacatccatttttcatctttgctcagtggccattgatagattttatgctatatgttacccattactttattccaccaaaataactattccagtcattaaaagattgctacttctatgttggtcggtccctggagcatttgccttcggggcggtcttctcagaggcctatgcagatggaatagagggctatgacatcttggttgcttgttccagttcctgcccagtgatgttcaacaagctatgggggaccaccttgtttatggcaggtttcttcactcctgggtctatgatggtggggatttacggcaaaatttttgcagtatccagaaaacatgctcatgccatcaataacttgcgagaaaatcaaaataatcaagtgaagaaagacaaaaaagctgccaaaactttaggaatagtgataggagttttcttattatgttggtttccttgtttcttcacaattttattggatccctttttgaacttctctactcctgtagttttgtttgatgccttgacatggtttggctattttaactccacatgtaatccgttaatatatggtttcttctatccctggtttcgcagagcactgaagtacattttgctaggtaaaattttcagctcatgtttccataatactattttgtgtatgcaaaaagaaagtgagtaggctttttctgca

SEQ ID NO: 2 - protein, 306 aa, Human
MYSFMAGSIFITIFGNLAMIISISYFKQLHTPTNFLILSMAITDFLLGFTIMPYSMIRSVENCWYFGLTFCKIYYSFDLMLSITSIFHLCSVAIDRFYAICYPLLYSTKITIPVIKRLLLLCWSVPGAFAFGAVFSEAYADGIEGYDILVACSSSCPVMFNKLWGTTLFMAGFFTPGSMMVGIYGKIFAVSRKHAHAINNLRENQNNQVKKDKKAAKTLGIVIGVFLLCWFPCFFTILLDPFLNFSTPVVLFDALTWFGYFNSTCNPLIYGFFYPWFRRALKYILLGKIFSSCFHNTILCMQKESE

SEQ ID NO: 3 - DNA, 915 bp, Human
  CDS (1)...(915)
atgtattcatttatggcaggatccatattcatcacaatatttggcaatcttgccatgataatttccatttcctacttcaagcagcttcacacaccaaccaacttcctcatcctctccatggccatcactgatttcctcctgggattcaccatcatgccatatagtatgatcagatcggtggagaactgctggtattttgggcttacattttgcaagatttattatagttttgacctgatgcttagcataacatccatttttcatctttgctcagtggccattgatagattttatgctatatgttacccattactttattccaccaaaataactattccagtcattaaaagattgctacttctatgttggtcggtccctggagcatttgccttcggggcggtcttctcagaggcctatgcagatggaatagagggctatgacatcttggttgcttgttccagttcctgcccagtgatgttcaacaagctatgggggaccaccttgtttatggcaggtttcttcactcctgggtctatgatggtggggatttacggcaaaatttttgcagtatccagaaaacatgctcatgccatcaataacttgcgagaaaatcaaaataatcaagtgaagaaagacaaaaaagctgccaaaactttaggaatagtgataggagttttcttattatgttggtttccttgtttcttcacaattttattggatccctttttgaacttctctactcctgtagttttgtttgatgccttgacatggtttggctattttaactccacatgtaatccgttaatatatggtttcttctatccctggtttcgcagagcactgaagtacattttgctaggtaaaattttcagctcatgtttccataatactattttgtgtatgcaaaaagaatag

SEQ ID NO: 4 - DNA, 831 bp, Mus musculus
  CDS (1)...(831)
atggcaggatccatattcatcacgatctttggcaacctggccatgatcatttccatttcctacttcaaacagcttcacacaccgaccaacctcctcattctctccatggccgtcaccgacttccttctcggattcaccatcatgccatatagtatggtcaggtcagtggaaaactgctggtatttcggacttacgttttgcaagatccattatagcttcgacctgatgcttagcataacgtccattttccacctttgctcggtggccgtcgatagattttatgccatctgtcaccctttgcattattgcaccaaaatgactatcccggtcgttaggcggctgctgctcgtctgctggtcagtccccggggcgtttgcctttggggtggtcttctccgaggcttacgctgatggaattgaaggctatgacattttggttgcatgttccagttcctgcccagtcatgttcaacaagctatgggggaccaccttgtttgtggcaggctttttcactcctagctcgatgatggtggggatttacggcaaaattttcgccgtatccaaaaaacacgctcgcgtaattgacaacttgccagaaaatcaaaacaatcaaatgaggaaggacaaaaaagcagccaaaactttagggatagtgatgggtgttttcttattgtgttggtttccatgtttcttcaccatcctgttagatccgtttctgaattcctctacgcctgcagttctgtttgatgccttaacatggttcggttattttaattccacatgtaatcccttaatttatggtttcttctaccttggtttcgcagagcactga

SEQ ID NO: 5 - protein, 276 aa, Mus musculus
MAGSIFITIFGNLAMIISISYFKQLHTPTNLLILSMAVTDFLLGFTIMPYSMVRSVENCWYFGLTFCKIHYSFDLMLSITSIFHLCSVAVDRFYAICHPLHYCTKMTIPVVRRLLLVCWSVPGAFAFGVVFSEAYADGIEGYDILVACSSSCPVMFNKLWGTTLFVAGFFTPSSMMVGIYGKIFAVSKKHARVIDNLPENQNNQMRKDKKAAKTLGIVMGVFLLCWFPCFFTILLDPFLNSSTPAVLFDALTWFGYFNSTCNPLIYGFFYLGFAEH